(19) United States
(12) Patent Application Publication
(10) Pub. No.: US 2023/0143212 A1
(43) Pub. Date: May 11, 2023

(54) Pharmaceutical Preparations Of Melatonin Suitable For Intranasal Administration

(71) Applicant: Navinta, LLC, Ewing, NJ (US)

(72) Inventors: Mahendra R. Patel, Delray Beach, FL (US); Bhaveshkumar Anilkumar Patel, Bensalem, PA (US)

(21) Appl. No.: 17/984,013

(22) Filed: Nov. 9, 2022

Related U.S. Application Data
(60) Provisional application No. 63/277,338, filed on Nov. 9, 2021.

(51) Int. Cl. A61K 9/08; A61K 9/00; A61K 47/26; A61K 47/38; A61K 31/4045; A61K 47/20

(52) U.S. Cl. CPC A61K 9/08; A61K 9/0043; A61K 31/4045; A61K 47/20; A61K 47/26; A61K 47/38

(57) ABSTRACT

A pharmaceutically acceptable intranasal spray formulation includes about 0.01% w/v to about 5% w/v melatonin, about 0% w/v to about 50% w/v liquid solvent selected from the group consisting of alcohol, polyethylene glycol, propylene glycol and combinations thereof, a pH system in an amount effective to maintain a pH of the formulation at about 4.5 to about 6.5, at least one preservative, and water. An aqueous formulation of melatonin for nasal administration as an in-situ gel includes melatonin, water, at least one organic solvent, and at least one gelling agent.

### FIELD OF THE INVENTION

The present invention relates to stable pharmaceutical preparation suitable for intranasal administration comprising melatonin.

### BACKGROUND OF THE INVENTION

There has been a recent resurgence in interest in the medical uses of melatonin. A plethora of studies have been published investigating great potential of melatonin in the treatment of sleep disorders. Based on the FDA Pharmacy Compounding Advisory Committee Meeting held on Jun. 9, 2021, Melatonin was nominated for inclusion on the list of bulk drug substances for use in compounding under section 503A of the Federal Food, Drug, and Cosmetic Act (FD&C Act). Melatonin is approved for use in Europe, Australia, and Japan, and many homeopathic preparations, and over the counter formulations of melatonin for oral use are readily available in USA.

Melatonin or N-acetyl-5-methoxytryptamine, is chemically named N-[2-(5-Methoxy-1H-indol-3-yl) ethyl] acetamide. Molecular formula: C₁₃H₁₆N₂O₂, Molecular weight: 232.3

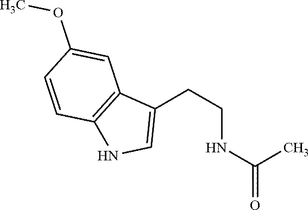 [SMILES: COc1ccc2[nH]cc(CCNC(C)=O)c2c1]

Melatonin is a naturally occurring hormone produced by the pineal gland and is structurally related to serotonin. In humans this hormone secretion increases soon after the onset of darkness, peaks between 2 and 4 a.m., and gradually falls during the second half of the night. Along with other neurotransmitters (e.g., serotonin, dopamine, norepinephrine), it plays an important role in the regulation of circadian rhythms that are responsible for the sleep-wake cycle. Abnormalities in the balance of this chemical messenger is frequently experienced by individuals, disrupting the normal sleep pattern. Jet lag, shift work syndrome, seasonal affective diseases, and sleep disturbances due to neurological, neurodevelopmental, psychiatric and behavioral disorders are amongst the typical causes for the circadian rhythm disorders. For the amelioration of the relevant clinical manifestations, the administration of exogenous melatonin has been proven as a safe and effective pharmacological option, efficiently restoring the levels of the nocturnal melatonin.

Melatonin is only slightly soluble in water (solubility of 2 mg/ml in water at 20° C.). It is soluble in organic solvents such as ethyl alcohol, DMSO, propylene glycol, polyethylene glycol, glycerol and dimethylformamide.

WO 86/05093 discloses a solution formulation of melatonin in 30% ethyl alcohol for the topical treatment of skin diseases. Use of ethyl alcohol in oral or intra nasal adult medicines is discouraged by regulatory agencies for obvious health reasons.

Lee et al. (Lee B-J et al. Arch. Pharm. Res. 1997, Vol.20, No.6, pages 560 to 565) have studied the solubility of melatonin in propylene glycol. According to the authors, the solubility of melatonin was enhanced in a mixture of propylene glycol and water at 25° C. (°C), increasing slowly up to 50 mM of melatonin (~12 mg/ml) as a function of propylene glycol concentration, until reaching 40%v/v propylene glycol and then steeply increased about 250 mM (60 mg/ml) at 60% v/v propylene glycol. In addition, it was shown that melatonin exhibits pH-independent solubility in water.

EP 3 530 289 disclosed a pharmaceutical solution for oral use comprising melatonin in 15% propylene glycol. Sucralose and strawberry flavor were used as sweetener and flavoring agent, respectively. As per Summary of Product Characteristics (SmPC) of Melatonin, 1 mg/ml oral solution (marketed by Colonis Pharma Ltd, UK) orally administered melatonin is almost completely absorbed. However, oral bioavailability is ~ 15%, owing to high first-pass metabolism where ~ 85% of the drug is metabolized. Plasma T_{½} is ~ 50 minutes, which necessitates repeated dosing of the drug for full night sleep.

Low oral bioavailability due to high first pass metabolism requires a melatonin formulation that can bypass the first pass metabolism. Moreover, as described in International Journal of Pharmaceutics Volume 182, Issue 1, 10 May 1999, Pages 1-5, as well as patent WO98/42333, the bioavailability of melatonin increased substantially in rabbits via intranasal route using a composition in polyethylene glycol-300 with and without bile salt as permeability enhancer. However, bile salt may cause irritation in intranasal mucosa.

The shorter half-life does not provide complete sleep up to 6-8 hours with available oral dosage form.

Nasal route is the most suitable route of administration to have a good bioavailability. Technically, an even bigger challenge is the development of stable, effective formulation which is suitable for chronic use. This is particularly relevant for pharmaceutical preparations that address chronic health problems such as sleep disorders. Moreover, melatonin is very prone to air oxidation. Thus, a significant issue arises when nasal preparation prone to atmospheric oxidation are provided in multi-dose containers because of the risk of degradation of the solution, once the closure system has been breached, due to repeated priming / repriming after first use by the patient. It is very difficult to formulate oxygen-sensitive drugs such as melatonin in multidose nasal spray device, which are sealed with a HDPE spray pump. Polymers are reasonably permeable to oxygen. Thus, oxidatively sensitive drugs require more than just oxygen exclusion during sealing to prevent oxidation from occurring. The present invention addresses these problems by providing pharmaceutical preparations of melatonin administered through nasal route which exhibit excellent shelf life with minimal excipients.

Patent application WO98/42333, CN1969814B, AU750361B2, CN105193794A and U.S. Pat. No. 6007834 disclose various types of melatonin formulations suitable for nasal administration, with cyclodextrin or glycerol.

A Chinese patent application CN1969814, mentions use of cellulose derivatives in the formulation of melatonin. However, it does not illustrate what specific compounds can be used for what effect.

In view of this background, it would be highly advantageous to provide a physically and chemically stable, safe formulation suitable for intranasal administration for the delivery of an optimized dose of melatonin to children and adults for the efficacious treatment of a sleep disturbances in autism spectrum disorders, as well as for the efficacious treatment of a sleep disturbances due to long traveling, stress, or other neurological conditions.

It is an object of the present invention to provide novel physically and chemically stable, safe formulations of melatonin suitable for intranasal administration for patients of age 2 years and above.

It is another objective of the present invention to provide novel pharmaceutical melatonin preparations suitable as sleep aid in patients with autism spectrum disorder.

It is another objective of the present invention to provide novel pharmaceutical melatonin preparations with longer half-life.

It is another objective of the present invention to provide novel pharmaceutical melatonin preparation suitable as sleep aid for the people suffering from sleep disorders.

These and other objectives, which will become apparent during the following detailed description, have been achieved by the discovery that certain formulations containing a solution of melatonin, a suspension of melatonin as well as in-situ gel of melatonin are effective for intranasal administration.

### SUMMARY OF THE INVENTION

To achieve at least some of the foregoing objectives, the present invention provides novel pharmaceutical preparations suitable for intranasal administration comprising melatonin with pharmaceutically acceptable excipients. In particular, pharmaceutical preparations containing Melatonin as an active ingredient suitable for intra-nasal administration and a pharmaceutically acceptable carrier include a mixture of alcohol, propylene glycol and polyethylene glycol 400 (PEG-400) individually or in a combination with water and pharmaceutically acceptable mucoadhesive excipients such as microcrystalline cellulose, carboxymethyl cellulose, hydroxy propyl methyl cellulose, and pectin. The pH of the pharmaceutical preparation is preferably adjusted to be in a range of about 4.5 to about 6.5. The pharmaceutical compositions of the present invention are stable for extended period and may contain preservatives. Pharmaceutical preparations are suitable as a sleep aid in patients in need thereof.

Thus, in a first aspect, the invention provides a pharmaceutical formulation in different forms which may be filled in suitable nasal spray device. The formulation includes:
(a) A solution including melatonin as active ingredient in admixture with one or more solubilizing agents selected from the group consisting of alcohol, propylene water, glycol, polyethylene glycol-400 (PEG-400), sorbitol, glycerin, mannitol and sucrose, and mixtures thereof;
b) A solution including melatonin as active ingredient in admixture with one or more solubilizing agents selected from the group consisting of alcohol, propylene glycol, water, polyethylene glycol-400 (PEG-400), mannitol and sucrose. Optionally, one or more viscosity enhancing agents selected from the group consisting of hydroxypropyl methylcellulose (HPMC), hydroxypropyl cellulose (HPC), hydroxyethyl cellulose (HEC) and mixtures thereof are added to the formulation;
c) A suspension comprising melatonin as active ingredient in admixture with one or more solubilizing agents selected from the group consisting of alcohol, propylene glycol, water, polyethylene glycol-400 (PEG-400), mannitol and sucrose. Optionally, one or more suspending agents selected from the group consisting of microcrystalline cellulose, carboxymethyl cellulose sodium and mixtures thereof are added to the formulation;
d) An in-situ gel including melatonin as active ingredient in admixture with one or more agents selected from the group consisting of alcohol, propylene glycol, water polyethylene glycol-400 (PEG-400), mannitol and sucrose and one or more gelling agents selected from the group consisting of poloxamer, pectin, and chitosan; or
e) A suspension including melatonin as active ingredient in admixture with alcohol as a solubilizing agent and one or more suspending agents selected from the group consisting of microcrystalline cellulose, carboxymethyl cellulose sodium and mixtures thereof.

It is understood that additional pharmaceutically acceptable excipients may be used in the above-mentioned formulations, in addition to those listed.

In some embodiments of these compositions, the active ingredient is melatonin.

In a second aspect, the present invention provides a process for preparing the above pharmaceutical formulations comprising one or more of the steps of:
(i) dissolving melatonin and one or more excipients in an organic solvent;
(ii) preparing a suspension by adding to the dissolved melatonin one or more suspending agents in water under continuous stirring;
(iii) preparing an in-situ gel by solubilizing one or more gelling agents;
(iv) adding the solubilizing agent; and
(v) combining a melatonin-solvent mixture with an aqueous base under continuous stirring.

In a third aspect, the present invention provides the above-mentioned melatonin compositions and their use as a medicament.

In a fourth aspect, the present invention provides a method for preventing and/or treating a sleep disorder in a patient, comprising administering a therapeutically effective amount of any of the above pharmaceutical compositions of melatonin.

The present invention may be useful for children and adult patients with autism spectrum disorder, who are more likely to suffer from sleeping disorders.

In a fifth aspect, the present invention provides a method for administration of a pharmaceutical composition comprising melatonin described herein using a spray device.

### DETAILED DESCRIPTION OF THE INVENTION

The present invention provides stable aqueous pharmaceutical solutions, suspensions or gels suitable for intranasal administration comprising melatonin in association with a pharmaceutically acceptable liquid carrier and viscosity enhancer.

The following detailed description is merely exemplary in nature and is not intended to limit the disclosed melatonin formulations, or any associated methods for producing or using the same described herein. Furthermore, there is no intention to be bound by any theory presented in the preceding background or the following detailed description.

The term “about” as used in connection with a numerical value throughout the specification and the claims denotes an interval of accuracy, familiar and acceptable to a person skilled in the art. In general, such interval of accuracy is ±10%. Thus, “about ten” means 9 to 11. All numbers in this description indicating amounts, ratios of materials, physical properties of materials, and/or use are to be understood as modified by the word “about,” except as otherwise explicitly indicated.

The intranasal formulations described in the present invention are useful to significantly lower the dose burden of melatonin by circumventing first-pass hepatic metabolism that is associated with oral dosage administration. Moreover, the intranasal formulations are useful where another route is ineffective, invasive, or impractical.

The melatonin compositions may contain water, alcohol, polyethylene glycol, propylene glycol individually or combination as solvent. In some embodiments, a suspension is formed with the help of suspending agent.

Typically, the concentration of melatonin is 0.1 mg/ml to 50 mg/mL. Melatonin can be used as a free base or in any pharmaceutically acceptable form and/or solvate thereof.

A melatonin solution may contain water, polyethylene glycol, propylene glycol, alcohol as individual or a combination of the four. The solvent is studied as an important liquid carrier and its level was studied and optimized in the course of the development.

As used throughout the present description and claims, a concentration of polyethylene glycol in melatonin nasal compositions may correspond to the concentration of polyethylene glycol-400. The concentration of polyethylene glycol-400 may be within the range of 0 mg/mL to 500 mg/mL and/or the concentration of propylene glycol may be withing the range of 0 mg/mL to 500 mg/mL.

As used throughout the present description and claims, the concentration of alcohol in melatonin nasal compositions may correspond to a concentration of ethyl alcohol. A concentration of ethyl alcohol may be in the range of 0 mg/mL to 500 mg/mL.

In some embodiments of the invention, sorbitol is used a stabilizing agent. A concentration of sorbitol is preferably within the range from 10 mg/ml to 250 mg/ml.

As used throughout the present description and claims, a concentration of sorbitol in the intranasal preparation of melatonin refers to a concentration of anhydrous sorbitol.

In some embodiments, glycerin is used as an alternative for a stabilizing agent. A concentration of glycerin is preferably within the range from 1 mg/ml to 200 mg/ml.

Tonicity of intranasal compositions may be adjusted between 200-600 mOsmol/L by adding salts or other tonicity adjusting agents.

In some embodiments of the invention, a tonicity agent such as mannitol, dextrose, and/or sucrose is used for preparation of isotonic intranasal solution. A concentration of tonicity agent is preferably within the range from 10 mg/ml to 200 mg/ml.

In certain embodiments of the invention, preservatives such as propylparaben, methylparaben, benzyl alcohol, benzalkonium chloride, or in form of any pharmaceutically acceptable salt thereof are used as preservatives in the intranasal composition.

It is well known that low drug loading may cause homogeneity problems for suspension dosage form. Surprisingly, it has been found that by proper selection of suspending agent at optimized level, it is possible to achieve content uniformity.

Suitable suspending agents include microcrystalline cellulose, carboxymethyl cellulose sodium and mixtures thereof.

If used, a concentration of suspending agent is preferably within the range from 0.01% w/v to 10% w/v.

The compositions of the present invention may be prepared using methods well-known in the art. For example, they may be prepared using the following process: half of the specified amount of purified water is added to the main vessel. The amount of solvent is charged under continuous stirring. The specified quantity of melatonin is transferred to the above solution and mixed until complete dissolution. The quantities of stabilizing agent, tonicity agent, antioxidant and preservatives are added sequentially. The pH is checked and, if required, adjusted to 4.5 - 6.5 with sodium hydroxide or hydrochloric acid. The volume of the solution is adjusted to the desired batch volume by adding purified water. The solution is filled into the designated containers, preferably under nitrogen purging.

As another example, the inventive compositions may be prepared using the following process: half of the specified amount of purified water is added to the main vessel. The amount of solvent is charged under continuous stirring. The specified quantity of melatonin is transferred to the above solution and mixed until complete dissolution. The quantities of suspending agent with or without viscosity enhancer, tonicity agent, antioxidant and preservatives are added sequentially. The pH is checked and, if required, adjusted to 4.5 - 6.5 with sodium hydroxide 0.1 N or hydrochloric acid 0.1 N. The volume of the solution is adjusted to the desired batch volume by adding purified water. The solution is filled into the designated containers, preferably under nitrogen purging.

In a particular embodiment of the invention, the pharmaceutical formulation comprises melatonin suspension to be sprayed in one or both nostrils.

In certain embodiments of the invention, the pharmaceutical formulation comprises a melatonin solution suitable to be sprayed in one or both nostrils.

In a preferred embodiment, a droplet particle size is 10 to 200 microns in diameter, as such size is appropriate for intranasal administration.

In a particular embodiment, an intranasal pharmaceutical preparation of melatonin solution includes melatonin as active ingredient, ethyl alcohol, propylene glycol, polyethylene glycol-400, water and optionally one or more tonicity adjusting agent, one or more stabilizer, antioxidant, and preservatives, wherein the pH of the intranasal solution is from 4.5 to 6.5.

In a particular embodiment of the invention, the intranasal pharmaceutical suspension includes melatonin as active ingredient, with or without propylene glycol, polyethylene glycol-400, one or more suspending agent, viscosity enhancer, one or more tonicity agent, antioxidant, and preservatives, wherein the pH of the intranasal solution is from 4.5 to 6.5.

In a particular embodiment of the invention, cellulose is used a suspending agent. The concentration is preferably within the range from 0.01% w/v to 10% w/v.

In a particular embodiment of the invention where cellulose is used a suspending agent, the cellulose is selected from the group consisting of microcrystalline cellulose and carboxymethyl cellulose.

In a particular embodiment of the invention, polymer (mucoadhesive polymer) is used as viscosity enhancer. A concentration of the polymer viscosity enhancer is preferably within the range from 0.01% w/v to 5% w/v. In some of those embodiments, the polymer is selected from the group consisting of hydroxypropyl methyl cellulose (HPMC), hydroxypropyl cellulose (HPC), hydroxyethyl cellulose (HEC), Pectin, Polyvinyl pyrrolidine (PVP).

In a particular embodiment of the invention, a tonicity agent is included, such as mannitol, dextrose, sucrose, for preparation of isotonic or hypertonic intranasal solution. A concentration of the tonicity agent is preferably within the range from 10 mg/ml to 200 mg/ml.

In a particular embodiment of the invention, preservatives such as propylparaben, methylparaben or in form of any pharmaceutically acceptable salts thereof are used as preservatives in the intranasal composition.

Also provided are melatonin thermo-reversible in-situ gel compositions to be administrated as drop in one or both nostrils. In addition to any of the above-described ingredients and combinations of ingredients and their respective amounts, the gel compositions comprise a thermo-reversible polymer.

A concentration of the thermo-reversible polymer is preferably within the range from 0.10% w/v to 40% w/v.

In a particular embodiment of the invention, poloxamer is used as the thermo-reversible polymer. In some embodiments, the grades of poloxamer are poloxamer 188 and poloxamer 407.

Polymer may also be used for a mucoadhesive effect. A concentration of the mucoadhesive polymer is preferably within the range from 0.01% w/v to 5% w/v.

In a particular embodiment of the invention, where a mucoadhesive polymer is used, the polymer is selected from the group consisting of hydroxypropyl methyl cellulose (HPMC), hydroxypropyl cellulose (HPC), hydroxyethyl cellulose (HEC).

In a particular embodiment of the invention, a tonicity agent such as mannitol, dextrose, or sucrose is used for preparation of an isotonic intranasal gel. A concentration of the tonicity agent is preferably within the range from 10 mg/ml to 200 mg/ml.

In a particular embodiment of the invention, preservatives such as propylparaben, methylparaben or in form of any pharmaceutically acceptable salt thereof are used in the intranasal gel composition.

The gel compositions of the present invention may be prepared using methods well-known in the art. For example, they may be prepared using the following process: half of the specified amount of purified water is added to the main vessel. The amount of poloxamer and solvent is charged under continuous stirring. The specified quantity of melatonin is transferred to the above solution and mixed until complete dissolution. The quantities of mucoadhesive polymer, tonicity agent, antioxidant and preservatives are added sequentially. The pH is checked and, if required, adjusted to 4.5 - 6.5 with sodium hydroxide 0.1 N or hydrochloric acid 0.1 N. The volume of the solution is adjusted to the desired batch volume by adding purified water. The solution is filled into the designated containers, preferably under nitrogen purging.

In a particular embodiment of the invention, the pharmaceutical formulation comprises melatonin gel to be administered as a drop in one or both nostrils.

### Experimental

The pharmaceutical compositions of melatonin described in the below table were formulated. A detailed procedure for preparation of each individual type of a composition is described below.

Compositions of Melatonin Formulation for Nasal Use:

| Composition Code | | F0 | F1 | F2 | F3 | F4 | F5 | F6 | F7 | F8 | F9 | F10 |
|---|---|---|---|---|---|---|---|---|---|---|---|---|
| sr.no | Material | mg/ mL | mg/ mL | mg/ mL | mg/ mL | mg/ mL | mg/ mL | mg/ mL | mg/ mL | mg/ mL | mg/ mL | mg/ mL |
| 1 | Melatonin | 10 | 10 | 10 | 10 | 10 | 10 | 10 | 10 | 10 | 10 | 10 |
| 2 | Ethyl Alcohol | ---- | ---- | ---- | ---- | ---- | ---- | ---- | ---- | ---- | ---- | 50 |
| 3 | Polyethylene Glycol | 200 | 200 | 200 | 200 | ---- | ---- | ---- | ---- | 200 | ---- | ---- |
| 4 | Propylene glycol | ---- | ---- | ---- | ---- | 150 | 150 | 150 | 150 | 150 | 150 | ---- |
| 5 | HPMC K4M | ---- | 1 | ---- | 1 | ---- | 1 | ---- | 1 | 0 | ---- | 1 |
| 6 | VIVAPUR® MCG* | ---- | 12.5 | 12.5 | ---- | ---- | 12.5 | 12.5 | ---- | 0 | ---- | ---- |
| 7 | Pectin | ---- | ---- | ---- | ---- | ---- | ---- | ---- | ---- | ---- | ---- | 10 |
| 8 | Sorbitol, Liquid | 70 | 70 | 70 | 70 | 70 | 70 | 70 | 70 | 70 | 70 | |
| 9 | Poloxamer 407 +188 (93:7) | ---- | ---- | ---- | ---- | ---- | ---- | ---- | ---- | ---- | 160 | --- |
| 10 | Mannitol | 42 | 42 | 42 | 42 | 42 | 42 | 42 | 42 | 0 | 42 | --- |
| 11 | Dextrose | ---- | ---- | ---- | ---- | ---- | ---- | ---- | ---- | ---- | ---- | 50 |
| 12 | Benzyl Alcohol | 9 | 9 | 9 | 9 | 9 | 9 | 9 | 9 | 9 | 9 | 9 |
| 13 | Methionine | 0.1 | 0.1 | 0.1 | 0.1 | 0.1 | 0.1 | 0.1 | 0.1 | 0 | 0.1 | 0.1 |
| 14 | HCl/NaOH | QS to pH 4.5-6.5 | | | | | | | | | | |
| 15 | Water (DI) | QS to 1 mL | | | | | | | | | | |

*(VIVAPUR® MCG 811 P) Microcrystalline Cellulose and Carboxymethylcellulose Sodium.

### Example 1: Melatonin Nasal Spray 10 mg/ml (Formulation F0)

4.2 g of mannitol and 7 g of sorbitol were dissolved in 50 mL of purified water. After getting a clear solution, 0.01 g of methionine was added and stirred until clear solution was formed.

In another vessel, 1 g of melatonin was dissolved in 20 g of PEG-400 and 0.9 g of benzyl alcohol was added to it. The aqueous solution prepared above was added to this solution under continuous stirring. The pH was adjusted to 4.5 to 6.5 using 0.1N HCl solution. Water was added to the mixture to make the total volume of the formulation to 100 mL and the formulation was filled into a multidose nasal spray device with the deliverable volume of 100 microliter per spray.

### Example 2 (Formulation F1): Melatonin Nasal Spray 10 mg/ml Suspension with Mucoadhesive Polymer

4.2 g of mannitol and 7 g of sorbitol were dissolved in 50 mL of purified water. After getting clear solution, 0.01 g of methionine was added and stirred until a clear solution was obtained. To the clear solution, 1.25 g of VIVAPUR MCG was added and stirred to get a homogenous liquid.

In another vessel, 1 g of melatonin was dissolved in 20 g of PEG-400 and 0.9 g of benzyl alcohol was added to it. Further, 0.1 g of HPMC was added under continuous stirring.

The aqueous mixture was added to the solvent phase. pH was adjusted to 4.5 to 6.5 using 0.1N NaOH solution. Water was added to the mixture to make the total volume to 100 mL. The formulation was filled in a suitable multidose nasal spray device with the deliverable volume of 100 microliter.

Other compositions described in the table (F2-F8) were prepared by analogous methods.

### Example 3 (Formulation F9): Melatonin Nasal Drop 10 mg/ml InSitu Thermo-Reversible Gel

In order to prepare a thermo-reversible gel, 15 g of Poloxamer 407 and 1 g of Poloxamer 188 were dissolved in 80 mL of cold water (temp 15-20° C.) and 0.9 g of benzyl alcohol was added to it. To this solution 4.2 g of mannitol and 7 g of sorbitol were added and stirred to dissolve. Melatonin solution was prepared by dissolving 1 g of melatonin in 15 g of PG.

The above aqueous solution was added to the solvent phase under continuous stirring. pH was adjusted to 4.5 to 6.5 using 0.1N HCl. Water was added to the mixture to make the total volume of the formulation to 100 mL. The formulation was filled into a suitable multidose nasal dropper device with the deliverable of 100 microliter per drop.

### Example 4 (Formulation F10): Melatonin Nasal Spray 10 mg/ml, Suspension

Melatonin (1 g) was dissolved in 5 g of ethyl alcohol. To this solution, 0.9 g of benzyl alcohol and 100 mg of HPMC were added.

In a separate vessel, 50 mL water was filled and 100 mg of pectin was dissolved by stirring for 4-5 hours at high speed. In another vessel, 5 g of dextrose was dissolved along with 625 mg of microcrystalline cellulose in 20 ml of water (DI). Both aqueous phases were mixed and stirred for 30 minutes.

The above aqueous phase was added into the solvent phase under continuous stirring. pH was adjusted between 4.5 to 6.5 using 0.1N NaOH solution. Volume was made up to 100 mL and the formulation was filled into a suitable multidose nasal dropper device with the deliverable of 100 microliter per drop.

### Example 5: Melatonin Nasal Spray 10 mg/ml, Solution

The solution compositions F11-F13 described in the table below were prepared according to the procedure of Example 1.

| Solution Composition | | F11 | F12 | F13 |
|---|---|---|---|---|
| Sr. No | Name of material | mg/mL | mg/mL | mg/mL |
| 1 | Melatonin | 10 | 10 | 10 |
| 2 | Propylene glycol | 120 | 120 | 120 |
| 3 | PEG 200 | 200 | 150 | 200 |
| 4 | Cellulose microcrystalline/ carboxymethyl cellulose sodium | 15 | --- | --- |
| 5 | PEG 4000 | -- | 100 | |
| 6 | Citric acid, | -- | 0.2 | 0.2 |
| | monohydrate | | | |
| 7 | Glycerin | 25 | 25 | 25 |
| 8 | Dextrose | 50 | 50 | 50 |
| 9 | Polysorbate 80 | 0.1 | 0.1 | 0.1 |
| 10 | Benzyl alcohol | 5 | 2 | 2.5 |
| 11 | HCl/NaOH | QS to pH | QS to pH | QS to pH |
| 12 | Purified Water | QS | QS | QS |

### Example 6: Melatonin Nasal Spray, a Suspension Formulation (Formulation: F14-F16)

The suspension formulations F14-F16 shown in the table below were prepared by the procedure of Example 4.

| Suspension composition | | F14 | F15 | F16 |
|---|---|---|---|---|
| Sr. No | Name of material | mg/g | mg/g | mg/g |
| 1 | Melatonin | 1 | 10 | 10 |
| 2 | Cellulose microcrystalline/ carboxymethylcellulose sodium | 20 | 20 | 20 |
| 3 | PEG 200 | --- | --- | 25 |
| 4 | Dextrose, Anhydrous | 50 | 50 | 50 |
| 5 | Benzalkonium Chloride | 0.3 | 0.3 | 0.3 |
| 6 | Polysorbate 80 | 0.08 | 0.08 | 0.08 |
| 7 | EDTA sodium | 0.1 | 0.1 | 0.1 |
| 8 | HCl/NaOH | QS to pH | QS to pH | QS to pH |
| 9 | DI water | QS to 1 g | QS to 1 g | QS to 1 g |

### Example 7: Animal Study

Test formulations F11 - F16 were used in an animal study to evaluate bioavailability of melatonin. Six New Zealand White Rabbits (3 male, 3 female) were used in each group, with a weight range between 1.8 to 2.5 kg in each study. All rabbits were dosed with 0.1 ml of melatonin test product intranasally using a plastic syringe in each nostril. Approximately 0.5-1 mL of blood was collected through marginal ear vein/central ear artery from each animal before dosing (pre dose) and at 0.083, 0.168, 0.25, 0.50, 0.75, 1, 2, 3, 4 & 6 hrs post dosing, in tubes containing K2/K3-EDTA. Plasma was separated using refrigerated centrifuge at 5000 rpm for 10 min and stored in -80° C. (±10° C.). The concentration of melatonin in collected rabbit plasma was determined by LC-MS/MS.

Concentration of Melatonin in Rabbit plasma after nasal administration:

| Group | Tmax | Cmax | AUC |
|---|---|---|---|
| | (hr) | (ng/mL) | (hr*ng/mL) |
| F11 | 0.168 | 543±195 | 205±57 |
| F12 | 0.083 | 461±191 | 186±74 |
| F13 | 0.083 | 411±155 | 202±70 |
| F14 | 0.095 | 124±33 | 40±6 |
| F15 | 0.11 | 1126±553 | 415±151 |
| F16 | 0.08 | 1163±302 | 373±84 |

The bioavailability of melatonin in rabbits after nasal administration of six formulations has been studied. In five out of six cases (F11, F12, F13, F15 and F16), a total amount of 2 mg/animal melatonin was administered and in formulation F14, a total amount of 0.2 mg/animal melatonin was administered. The results are reported in the above table. In each case, significant melatonin was found in rabbit’s plasma after administration via nasal route.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the subject matter in any way. Rather, the foregoing detailed description will provide those skilled in the art with a convenient road map for implementing an exemplary embodiment, it being understood that various changes may be made in the function and arrangement of elements described in an exemplary embodiment without departing from the scope as set forth in the appended claims and their legal equivalents.

What is claimed is:

1. A pharmaceutically acceptable intranasal spray formulation comprising:
about 0.01 % w/v to about 5% w/v melatonin;
about 0% w/v to about 50% w/v liquid solvent selected from the group consisting of alcohol, polyethylene glycol, propylene glycol and combinations thereof;
a pH system in an amount effective to maintain a pH of the formulation at about 4.5 to about 6.5;
at least one preservative; and
water.

2. The formulation of claim 1, further comprising a stabilizer.

3. The formulation of claim 2, wherein the stabilizer comprises sorbitol and/or glycerin.

4. The formulation of claim 2, comprising about 1% w/v to about 25% w/v of the stabilizer.

5. The formulation of claim 2, comprising about 0.1% w/v to about 20% w/v of the stabilizer.

6. The formulation of claim 1, further comprising a tonicity agent.

7. The formulation of claim 6, wherein the tonicity agent is selected from the group consisting of mannitol, dextrose, sucrose, and combinations thereof.

8. The formulation of claim 6, comprising about 1% w/v to about 20% w/v of the tonicity agent.

9. The formulation of claim 1, further comprising an antioxidant.

10. The formulation of claim 9, wherein the antioxidant comprises methionine.

11. The formulation of claim 9, comprising about 0.02% w/v to about 0.2% w/v of the antioxidant.

12. The formulation of claim 1, wherein the preservative is selected from benzyl alcohol, methyl paraben and propylparaben.

13. A melatonin nasal spray composition comprising:
melatonin,
water, and
at least one organic solvent selected from propylene glycol, polyethylene glycol, ethanol, and glycerol.

14. The composition of claim 12, comprising about 0.01% w/v to about 5% w/v melatonin.

15. The composition of claim 12, comprising about 0.1% w/v to about 50% w/v of the at least one organic solvent.

16. The composition of claim 12, further comprising a preservative selected from benzyl alcohol, methyl paraben, and propylparaben.

17. A pharmaceutically acceptable intranasal spray formulation for a sustained release or immediate release with a sustained release effect of melatonin comprising:
about 0.01 % w/v to about 5% w/v melatonin;
about 10% w/v to about 50% w/v solvent comprising alcohol, polyethylene glycol, propylene glycol or combinations thereof;
about 1 % w/v to about 25% w/v sorbitol or glycerin;
about 1 % w/v to about 20% w/v mannitol, dextrose, or sucrose;
about 0.01% w/v to about 10% w/v microcrystalline cellulose and/or carboxymethyl cellulose;
at least one preservative selected from benzyl alcohol, methyl paraben and propylparaben; and
about 0.02% w/v to about 0.25% w/v methionine.

18. An aqueous formulation of melatonin for nasal administration as an in-situ gel comprising:
melatonin;
water;
at least one organic solvent; and
at least one gelling agent.

19. The formulation of claim 18, wherein the at least one gelling agent comprises a thermoreversible polymer.

20. The formulation of claim 18, comprising from about 0.10% w/v to about 40% w/v of the at least one gelling agent.
